(19) United States
(12) Patent Application Publication
(10) Pub. No.: US 2012/0302645 A1
(43) Pub. Date: Nov. 29, 2012

(54) USE OF HYDROXYTYROSOL FOR IMPROVING MUSCLE DIFFERENTIATION

(76) Inventors: Jiankang Liu, Shan'xi (CN); Ying Wang-Schmidt, Shanghai (CN); Karin Wertz, Rhinefelden (DE); Zhihui Feng, Shan'xi (CN)

(21) Appl. No.: 13/500,740

(22) PCT Filed: Oct. 8, 2010
(86) PCT No.: PCT/CN2010/001550

§ 371 Date: Jun. 25, 2012

Related U.S. Application Data
(60) Provisional application No. 61/272,578, filed on Oct. 7, 2009.

(51) Int. Cl. A61K 31/05; A61P 29/00; C07C 39/11; A61P 21/00

(52) U.S. Cl. 514/731; 568/766

(57) ABSTRACT

The use of hydroxytyrosol or an olive juice extract containing hydroxytyrosol for improving muscle differentiation and thus improving or maintaining the body's adaptation to exercise is disclosed. The pharmaceutical and nutraceutical compositions comprising hydroxytyrosol useful for conditions characterized by altered muscle differentiation especially under inflammatory conditions, such as delayed onset muscle soreness subsequent to strenuous exercise or sarcopenia are provided.

### FIELD OF THE INVENTION

This invention is related to the use of hydroxytyrosol (“HT”), or an olive juice extract containing hydroxytyrosol as an agent to improve muscle differentiation and thus improve or maintain the body's adaptation to exercise. It also relates to pharmaceutical and nutraceutical compositions useful for conditions characterized by altered muscle differentiation especially under inflammatory conditions, such as delayed onset muscle soreness subsequent to strenuous exercise or sarcopenia.

### BACKGROUND OF THE INVENTION

Muscle differentiation, i.e. the differentiation of satellite cells into new muscle fibers (myofibers, myotubes), plays a central role in mediating the growth and regeneration of skeletal muscle both during postnatal growth and in adult life.

Satellite cells are a heterogeneous population composed of stem cells and committed myogenic progenitors. Satellite cells uniformly express the transcription factor Pax7, and Pax7 is required for satellite cell viability and to give rise to myogenic precursors that express the basic helix-loop-helic (bHLH) transcription factors Myf5 and MyoD. Pax7 activates expression of target genes such as Myf5 and MyoD through recruitment of the Wdr5/Ash2L/MLL2 histone methyltransferase complex. Extensive genetic analysis has revealed that Myf5 and MyoD are required for myogenic determination, whereas myogenin and MRF4 have roles in terminal differentiation.

Muscle differentiation is required for maintenance of the skeletal musculature, for wound healing after surgery, trauma or strenuous exercise. Moreover, the formation of new muscle fibers (myotubes) is required for muscle growth.

Improving or maintaining muscle differentiation is needed e.g. for adaptation to exercise, especially to resistance exercise, and thus is important for sports performance. Muscle differentiation is also needed for mobility and all associated aspects of health, ability to work, and to lead an active life style.

Improved muscle differentiation is a particular need of elite athletes, whose professional success depends on an optimized training regimen to be able to perform at top level at times of important competitions. Moreover, healthy muscle differentiation is of interest for life style athletes (recreationally active people, weekend warriors), who harness important experiences of fun and satisfaction from successful exercise performance. Women, who in general have a lower muscle mass than men, often are concerned about their physical capabilities, hence are in need of good muscle differentiation.

A successful training regimen strives to optimize adaptation of the body to exercise. Adaptation to exercise among others includes an increase in aerobic exercise capacity, increased lipid storage especially in oxidative muscle fibers, activation of the endogenous antioxidant defense system, increased vascularization of the musculature, increased erythropoiesis, synthesis of contractile fibers within muscle cells such as actin and myosin and others, and the recruitment of satellite cells to differentiate and fuse into myotubes.

Oxidative stress induced by exercise is thought to be causally involved in inducing adaptation to exercise, i.e. successful training. The reactive oxygen species are generated during muscle contractions, but also during aerobic energy metabolism (oxidative phosphorylation, oxphos, aerobic respiration). The redox-sensitive MAPK and NFkB signaling pathways and the resulting reactions of cellular stress and inflammation are regarded as important pathways mediating adaptation to exercise (reviewed in Li Li Ji, Free Radical Biology & Medicine 44 (2008), 142-152, Li Li Ji Exp Gerontology 42 (2007), 582-593). In line with this, intervention studies with the antioxidants allopurinol and vitamin C in animal models and in humans have found that antioxidant supplementation reduced adaptation to exercise (Gomez-Carbrera et al. 2005 J Physiol 567, 113-120, Gomez-Carbrera et al. 2008 Am. J. Clin. Nutr. 87(1):142-149, Ristow et al (2009) PNAS 106, 8665-8670).

TNFa is a known mediator of inflammation, which activates NFkB signaling. While hydroxytyrosol has been shown to be an inhibitor of NFkB signaling in the monocyte cell line THP-1 and in primary monocytes and monocyte-derived macrophages (Zhang et al 2009 Biol. Pharm. Bull. 32(4) 578-582; Brunelleschi et al, 2007 Pharmacological Research 56: 542-549), it is not at all clear that it would also display this ability in muscle cells. For example, Baudy et al., Int Immunopharmacol. 2009 September; 9(10):1209-14. Epub 2009 July 21, which is hereby incorporated by reference, have shown that for EGCG and FGF, inhibition of NFkB in muscle cells cannot be extrapolated from the ability of a product/compound to inhibit NFkB in other cell types.

An antioxidant is a molecule capable of slowing or preventing the oxidation of other molecules. Antioxidants terminate oxidation chain reactions by removing free radical intermediates, and inhibit other oxidation reactions by being oxidized themselves. Reducing agents such as thiols or polyphenols often exert antioxidant property. Well known antioxidants such as Vitamins A, C and E scavenge free radicals and protect DNA, proteins and lipids from damage. Antioxidants also protect mitochondria from reactive oxygen species and free radicals generated during ATP production.

Furthermore, improved muscle differentiation can help alleviate or prevent muscle loss during inactivity, chronic illness or aging (sarcopenia), thus helping to preserve independent living and quality of life. Sarcopenia is a disorder of progressive muscle loss, usually occurring in old age.

However, it is believed that athletes should avoid the ingestion of anti-oxidants as it is believed this inhibits the breakdown/build up cycle of muscle growth.

### DETAILED DESCRIPTION OF THE INVENTION

It has been surprisingly found, in accordance with this invention, that hydroxytyrosol (“HT”) can improve muscle differentiation, especially in inflammatory conditions, such as after strenuous exercise or during other inflammatory muscle conditions, such as sarcopenia.

Thus one aspect of this invention is a method of maintaining or improving muscle differentiation comprising administering an effective amount of hydroxytyrosol to a mammal, and observing a muscle differentiation effect. Preferably the mammal is a human, and even more preferably the human is an elete athlete, or at the other end of the spectrum, a person who exhibits or is likely to exhibit symptoms of sarcopenia.

Hydroxytyrosol (3,4-dihydroxyphenylethanol) may be of synthetic origin or it may be isolated from extracts of olive leaves, olive fruits, olive pulp, or vegetation water of olive oil production. Thus, the term “hydroxytyrosol” also encompasses any material or extract of a plant or any material or extract of parts of a plant or any extract/concentrate/juice of fruits of a plant (such as olives) containing it, especially in an amount of at least 1.5 weight %, preferably in an amount of at least 30 weight %, and more preferably in an amount of at least 40 weight-%, more preferably in an amount of at least 50, 55, 60, 65, 70, 75, 80, 85, 90 weight-%, and most preferably in an amount of at least 45 weight-%, based on the total weight of the plant material or extract. The commercial form of the extract may or may not be standardized to lower concentrations of hydroxytyrosol by formulating the hydroxytyrosol with suitable formulation excipients. The terms “material of a plant” and “plant material” used in the context of the present invention means any part of a plant, also the fruits.

In further embodiments of the present invention, hydroxytyrosol derivatives such as esters and physiologically/pharmaceutically acceptable salts may be used instead of or in addition to hydroxytyrosol. It is also possible to use a mixture of hydroxytyrosol and hydroxytyrosol derivatives. Derivatives can be e.g. esters or glucosides, and are known to the person skilled in the art. Preferred esters of hydroxytyrosol are e.g. acetates or glucuronide conjugates; as well as oleuropein being the most preferred one.

Thus, one aspect of this invention is the use of hydroxytyrosol in the manufacture of a medicament or food product (for humans and/or animals) which is useful for maintaining or increasing muscle differentiation or muscle growth or for reducing or balancing muscle loss. Another aspect of this invention is a method of maintaining or increasing muscle differentiation or muscle growth or of reducing or balancing muscle loss in a subject in need thereof comprising administering a muscle differentiation-inducing or stimulating amount of hydroxytyrosol, and observing muscle differentiation.

Another aspect of this invention is the use of hydroxytyrosol in the manufacture of a medicament or food product (for humans and/or for animals) which is useful for maintaining or increasing muscle differentiation or muscle growth or for reducing or balancing muscle loss. These products help to ensure normal muscle function and to help improve the body's adaptation to exercise.

Another aspect of this invention are nutraceuticals which comprise a muscle differentiation-inducing amount of hydroxytyrosol.

“Observing muscle differentiation” means that the person who administered the HT or the person ingesting the HT notices a difference in muscle differentiation. This may be manifested in the person noticing that he/she adapts to exercise better, feels better after exercise compared to exercising without ingesting HT, and experiences less DOMS (delayed onset muscle soreness). The person or a trainer or other third party notices that the person ingesting HT responds better to training than before, or in comparison to a person of similar age, sex and fitness level who does not ingest HT.

“Elite athlete” refers to an athlete who spends at least 10 hours per week in a training regime.

“Overtraining” takes place when a person spends at least 10% more time per week training than is the usual average. It may take place prior to an important sporting event.

“Strenuous exercise” has various biochemical markers which can be measured. For example, microlesions can occur in the myotubes. Additionally, while it is appreciated that exercise in general can lead to a downregulation of lymphocytes, in a strenuous exercise situation, lymphocytes are down-regulated at least 25% more than in normal exercise. Further, there is an upregulation of creatinine levels to at least 10% more than is seen in normal exercise. Other marlers which are increased at least 10% above that observed in a normal exercise situation are lactate dehydrogenase and creatinine kinase.

When used, hydroxytyrosol has the following benefits:
helps improve effectiveness of your training regimen
helps prevent symptoms of overtraining,
helps reduce delayed onset muscle soreness (DOMS),
helps improve your training outcome after strenuous exercise,
helps your body adapt to exercise better,
helps you to be able to train harder,
helps you reduce the risk to overtrain,
helps improve muscle regeneration after exercise especially strenuous exercise,
helps improve muscle growth after exercise, especially strenuous exercise,
helps muscle regeneration in aching muscles,
supports muscle growth after strenuous exercise,
supports muscle maintenance in elderly,
supports muscle maintenance in Duchenne muscle dystrophy,
supports muscle maintenance in inflammatory muscle wasting conditions

### BRIEF DESCRIPTION OF THE FIGURES

FIG. 1 Hydroxytyrosol increases protein expression of myosin heavy chain. At the initiation of differentiation, C2C12 myoblasts were pre-treated with hydroxytyrosol at concentrations of 0, 1, 5, 10 or 50 microM) in differentiation medium for 30 minutes, and then were co-cultured with TNF-α (10 ng/ml) in differentiation medium for 5 days. Final results were presented as percentage of control. Data are mean±SE (n=2). MHC expression was determined by Western Blotting.

FIG. 2 Hydroxytyrosol increases protein expression of myogenin. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 5 days. Final results were presented as percentage of control. Data are mean±SE (n=2). Myogenin expression was determined by Western Blotting.

FIG. 3 Hydroxytyrosol increases creatine kinase activity. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 4 or 5 days. Final results were presented as percentage of control. Data are mean±SE (n=2). Creatine kinase is a muscle cell-specific enzyme, thus activity was measured as a marker of muscle cell differentiation.

FIG. 4 Hydroxytyrosol increases protein expression of PGC1α. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 4 or 5 days. Final results were presented as percentage of control. Data are mean±SE (n=2). PGC1α is a key transcriptional regulator of mitochondrial biogenesis (thus aerobic energy generation capacity), and is also involved in muscle differentiation by coactivating MEF2 and PPARδ, which regulate muscle differentiation and fiber type switching towards a more aerobic phenotype (red, slow-twitch, high endurance type I fibers).

FIG. 5 Hydroxytyrosol increases protein expression of mitochondrial complexes I and II. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 4 or 5 days. Final results were presented as percentage of control. Data are mean±SE (n=2). Mitochondrial complexes I and II are indirect transcriptional targets of PGC1α and a marker of mitochondrial biogenesis (thus aerobic energy generation capacity).

FIG. 6 Hydroxytyrosol rescues muscle differentiation suppressed by the inflammatory cytokine TNFα. At the initiation of differentiation, C2C12 myoblasts were pre-treated with Hydroxytyrosol at concentrations of 0 or 1 microM in differentiation medium for 30 minutes, and then were co-cultured with TNF-α (10 ng/ml) in differentiation medium for 5 days. Light microscopy of C2C12 cell cultures.

FIG. 7 Hydroxytyrosol does act as an antioxidant in C2C12 myoblasts treated with TNFa, and against current teaching nevertheless induces molecular pathways connected with improved adaptation to exercise. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 5 days. Final results were presented as percentage of control. Data are mean±SE (n=2).

FIG. 8 Hydroxytyrosol increases protein expression and activity of mitochondrial complexes I and II. At the initiation of differentiation, C2C12 myoblasts were pre-treated with Hydroxytyrosol at concentrations of 0, 1, 5, 10 or 50 microM in differentiation medium for 30 minutes, and then were co-cultured with TNF-α (10 ng/ml) in differentiation medium for 5 days. Final results were presented as percentage of control. Data are mean±SE (n=2). Mitochondrial complex I is a marker for the mitochondrial capacity for oxidative phosphorylation (oxphos, aerobic energy metabolism) and an indirect transcriptional target of PGC1a. Increased mitochondrial capacity is an important aspect of the body's adaptation to exercise.

FIG. 9 Effect of HT supplement and LTE on endurance capacity and muscle atrophy. SD rats were given either saline or treated with HT (25 mg/kg/day) in both sedentary and exercise groups. (Sed for sedentary, Exe for long-term endurance exercise; Sed+HT for sedentary with 25 mg/kg HT treatment, and Exe+HT for LTE with 25 mg/kg HT treatment). After 8 weeks, rats were run to exhaustion on a treadmill, and run time was recorded as endurance capacity (A). Skeletal muscle mRNA was extracted and Atrogin-1 and MuRF1 were analyzed by real time PCR (B). Values are means±S.E.M from 10 rats; ̂̂p<0.01 vs. Sedentary control; *p<0.05, **p<0.01 vs. exercise control.

FIG. 10. Effect of HT supplement and LTE on autophagy activation. SD rats were given either saline or treated with HT (25 mg/kg/day) in both sedentary and exercise groups. (Sed for sedentary, Exe for long-term endurance exercise; Sed+HT for sedentary with 25 mg/kg FIT treatment, and Exe+HT for LTE with 25 mg/kg HT treatment). After 8 weeks, rats were scarified and autophagy related proteins Atg7, Beclin-1, LC3B were determined by Western blot (A Western image, B statistical results); skeletal muscle mRNA was prepared and FoxO3 mRNA level was analyzed by real time RT-PCR (C). Values are means±S.E.M from 10 rats; ̂p<0.05 vs. sedentary control; *p<0.05, **p<0.01 vs. exercise control.

FIG. 11. Effect of HT supplement and LTE on mitochondria content. SD rats were given either saline or treated with HT (25 mg/kg/day) in both sedentary and exercise groups. (Sed for sedentary, Exe for long-term endurance exercise; Sed+HT for sedentary with 25 mg/kg HT treatment, and Exe+HT for LTE with 25 mg/kg HT treatment). After 8 weeks, rats were sacrified and muscle mitochondria subunits expression and PGC-1α were determined by Western blot (A Western image, B statistical results of PGC-1α and complex I subunit level). Mitochondrial DNA number or NRF1 and Tfam RNA level were analyzed by real time PCR or RT-PCR, respectively (C). Values are means±S.E.M from 10 rats; ̂p<0.05 vs. sedentary control; *p<0.05 vs. exercise control.

FIG. 12. Effect of HT supplement and LTE on mitochondria dynamics. SD rats were given either saline or treated with HT (25 mg/kg/day) in both sedentary and exercise groups. (Sed for sedentary, Exe for long-term endurance exercise; Sed+HT for sedentary with 25 mg/kg HT treatment, and Exe+HT for LTE with 25 mg/kg HT treatment). After 8 weeks, rats were sacrified and muscle mitochondria dynamics-related proteins Drp1, Mfn1, Mfn2 were determined by Western blot (A Western image, B statistical results); mitochondrial were isolated and complex I and II activities were analyzed (C). Values are means±S.E.M from 10 rats; ̂p<0.05 vs. sedentary control; *p<0.05 vs. exercise control.

FIG. 13. Effect of HT supplement and LTE on oxidative status. SD rats were given either saline or treated with HT (25 mg/kg/day) in both sedentary and exercise groups. (Sed for sedentary, Exe for long-term endurance exercise; Sed+HT for sedentary with 25 mg/kg HT treatment, and Exe+HT for LTE with 25 mg/kg HT treatment). After 8 weeks, rats were sacrified and oxidative stress response pathway activations in muscle were determined by Western blot (A); gene expression of p53, p21, and MnSOD were determined by Western blot (B Western blot image, C statistical results). Values are means±S.E.M from 10 rats; ̂p<0.05, ̂̂p<0.01 vs. sedentary control; *p<0.05, **p<0.01 vs. exercise control.

FIG. 14. Effect of HT supplement and LTE on the immune system. SD rats were given either saline or treated with HT (25 mg/kg/day) in both sedentary and exercise groups. (Sed for sedentary, Exe for long-term endurance exercise; Sed+HT for sedentary with 25 mg/kg HT treatment, and Exe+HT for LTE with 25 mg/kg HT treatment). After 8 weeks, one day before and after the endurance capacity test, blood was collected twice for testing BUN level, (A), WBC number (B), LYM level (C), and CREA level (D) Values are means±S.E.M from 10 rats; ̂p<0.05, ̂̂p<0.01 vs. sedentary control; *p<0.05, **p<0.01 vs. exercise control.

The inventors have also demonstrated that hydroxytyrosol at 1.0-10 μM increases muscle differentiation under inflammatory conditions as found e.g. but not exclusively after strenuous exercise. Further, hydroxytyrosol can thus maintain tissue function and prevent tissue failure triggered by insufficient muscle differentiation/regeneration. Thus, another aspect of this invention is the use of HT to protect muscle during strenuous exercise.

During periods of strenouous exercise, muscle can become damaged due to microlesions which form in the mycotubes. This can lead to inflammation, and to DMOS (delayed onset of muscle soreness). Overtraining and overexertion are primary causes of DMOS. Even in experienced or elite athletes DMOS can be a problem. Thus, another aspect of this invention is a method of preventing or lessening DMOS comprising administering HT before, during, or immediately after incurring mycotubal damage, and observing a lessening of DMOS. Another aspect of this invention is administering HT in order to maintain creatinine levels at levels which are within 25% of baseline (levels at rest), preferably within 10%.

Those which can benefit from maintaining or increasing muscle differentiation include:
A. Elite athletes
B. Lifestyle athletes
C. Individuals with inflammatory muscle disorders such as sarcopenia, Duchenne muscle dystrophy, “Weichteilrheuma”, inflammatory muscle wasting disorders, chronic muscle inflammation (myositis)
D. Domestic animals including pets, especially dogs, cats, horses, and racing camels.

### Formulations

Hydroxytyrosol or olive juice extracts containing hydroxytyrosol according to the present invention can be used in any suitable form such as a food, or a beverage, as Food for Special Nutritional Uses, as a dietary supplement, as a nutraceutical or in animal feed or food.

The hydroxytyrosol or olive juice/leaf extracts containing hydroxytyrosol may be added at any stage during the normal process of these products. Suitable food products include e.g. cereal bars, bakery items such as cakes and cookies or other types of snacks such as chocolate, nuts, gummy bears, chewing gums, and the like, and also liquid foods such as soups or soup powders, and dairy products, such as dairy shots and yoghurt. Suitable beverages encompass non-alcoholic and alcoholic drinks as well as liquid preparations to be added to drinking water and liquid food. Non-alcoholic drinks are preferably mineral water, sport drinks, energy drinks including those containing glucuronolactone for increased mental alertness and taurine for detoxification, hybrid energy drinks, near water drinks, fruit juices, lemonades, smoothies, teas, instant beverages, and concentrated drinks such as shots and mini-shots. The sports drinks can be hypotonic, hypertonic or isotonic. Sports drinks can be available in liquid form, as concentrates or as powder (to be dissolved in a liquid, as for example water). Examples of Foods for Special Nutritional Uses include the categories of sport food (e.g. sports nutrition formulations such as protein shots, protein powder, gels and the like), slimming foods, infant formula and clinical foods. Feed includes any animal food or feed premix, including items such as pet treats and snacks.

The term “dietary supplement” as used herein denotes a product taken by mouth that contains a compound or mixture of compounds intended to supplement the diet. The compound or mixture of compounds in these products may include: vitamins, minerals, herbs or other botanicals and amino acids. Dietary supplements can also be extracts or concentrates, and may be found in many forms such as tablets, capsules, softgels, gelcaps, liquids, or powders. The dietary supplement can also be used to promote energy to the dermal mitochondria, thus enhancing esthetic qualities of the skin.

The term “nutraceutical” as used herein denotes the usefulness in both the nutritional and pharmaceutical field of application. The nutraceutical compositions according to the present invention may be in any form that is suitable for administrating to the animal body including the human body, especially in any form that is conventional for oral administration, e.g. in solid form such as (additives/supplements for) food or feed, food or feed premix, tablets, pills, granules, dragées, capsules, and effervescent formulations such as powders and tablets, or in liquid form such as solutions, emulsions or suspensions as e.g. beverages, pastes and oily suspensions. Controlled (delayed) release formulations incorporating the hydroxytyrosol or olive juice extracts containing hydroxytyrosol according to the invention also form part of the invention. Furthermore, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns. The nutraceutical can further comprise usual additives, for example sweeteners, flavors, sugar, fat, emulgators, preservatives. The nutrition can also comprise other active components, such as (hydrolyzed) proteins as described in for example WO 02/45524. Also anti-oxidants can be present in the nutrition, for example flavonoids, carotenoids, ubiquinones, rutin, lipoic acid, catalase, glutatione (GSH) and vitamins, such as for example C and E or their precursors.

Generally between about 1 mg to about 500 mg of hydroxytyrosol in an olive extract is effective per serving. Preferably between 1 mg and 250 mg hydroxytyrosol is present in the olive extract, and even more preferably between about 1 mg and 100 mg in an olive extract is used

The daily dosage of hhydroxytyrosol for humans (70 kg person) may be at least 0.1 mg. It may vary from 1 to 500 mg, preferably from 5 to 100 mg.

The preferred dose of hydroxytyrosol varies from 0.28 to 1.9 mg/kg metabolic body weight for mammals, whereby

“metabolic body weight” [in kg]=(body weight [in kg])^{0.75}

for mammals. That means e.g. that for a human of 70 kg the preferred daily dose would vary between 6.77 and 45.98 mg, for a 20 kg dog the preferred daily dose would vary between 2.23 and 15.1 mg.

The following non-limiting Examples are presented to better illustrate the invention.

### EXAMPLES

### Example 1

### Materials and Methods

### Materials

Bovine serum albumin (BSA-fatty acid free), 1,4-dithio-DL-threitol (DTT), and ATP. Bioluminescent Assay Kit were obtained from Sigma (St. Louis, Mo., USA); 2′, 7′-Dichlorodihydrofluorescein diacetate (H₂DCF-DA) from Calbiochem (Darmstadt, Germany); TRIzol from Invitrogen (Carlsbad, USA); Reverse Transcription System kit and SYBR Green from Promega (Manheim, Germany); HotStarTaq from TaKaRa (Otsu, Shiga, Japan), Anti-oxphos complex I, II, from Invitrogen (Carlsbad, Calif., USA), Ppargc1a, 18S rRNA and β-actin primers were synthesised by Bioasia Biotech (Shanghai, China).

C2C12 Cell Culture and Treatments with TNFα

Mouse C2C12 myoblasts were purchased from ATCC (Manassas, Va., USA) and maintained in Dulbecco's modified Eagle's medium supplemented with 10% fetal bovine serum (Invitrogen) at a confluence of 60-70%. To initiate differentiation, cells were allowed to reach 100% confluence, and medium was changed to Dulbecco's modified Eagle's medium containing 2% horse serum (Invitrogen) and changed every 2 days. Full differentiation with myotube fusion and spontaneous twitching was observed at 8 days. Cells were pretreated with HT for 24 in growth medium, and then induced with TNF (10 ng/ml) in differentiation medium for 4 days.

### Western Blot Analysis

After treatment, cells were washed twice with ice-cold PBS, lysed in sample buffer (62.5 mM Tris-C1 pH 6.8, 2% SDS, 5 mM DTT) at room temperature and vortexed. Cell lysates were then boiled for 5 minutes and cleared by centrifugation (13,000 rpm, 10 minutes at 4° C.). Protein concentration was determined using Bio-Rad DC protein assay. The soluble lysates (10 μg per lane) were subjected to 10% SDS-PAGE, proteins were then transferred to nitrocellulose membranes and blocked with 5% non-fat milk/TBST for 1 h at room temperature. Membranes were incubated with primary antibodies directed against myosin heavy chain (MHC) (1:1000), myogenin (1:2000), Complex I (1:2000), PGC-1α (1:1000), α-tubulin (1:50 000) in 5% milk/TBST at 4° C. overnight. After washing membranes with TBST three times, membranes were incubated with horseradish peroxidase-conjugated secondary antibody for 1 h at room temperature. Western blots were developed using ECL (Roche Manheim, Germany) and quantified by scanning densitometry (Boudina et al., 2005).

### Measurement of Creatine Kinase Activities (CK)

CK activities and GSH content were determined using the CK detection kit (Jiancheng Bioengineering Institute, Nanjing, China).

### Assessment of ROS Production

ROS level in C2C12 cells was monitored by 2′,7′-Dichlorodihydrofluorescein diacetate (H2DCFH-DA) (Voloboueva et al., 2005). Briefly, 2*10⁶ cells were used. After isolation, C2C12 were incubated with 25 μM DCFH-DA (previously dissolved in DMSO, 0.1% DMSO final concentration) for 30 min at 37° C. At the end of the incubation, cells were washed three times with PBS, and then fluorescence was analyzed by flow cytometry (FACS Calibur Becton Dickinson).

### Statistical Analysis

Data from three separate experiments are presented as means±SE. Statistical significance was determined by using one-way ANOVA with Students' T-Tests between the two groups. The criterion for significance was set at **p<0.01, *p<0.05 and #p<0.05.

### Results

Effects of Hydroxytyrosol on Protein Expression of MHC and Myogenin During Myogenic Differentiation in C2C12 Cells Treated with TNF-α.

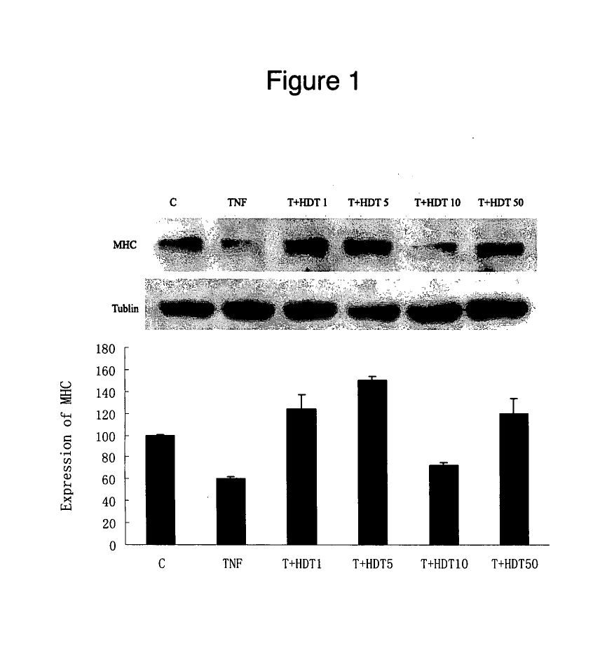

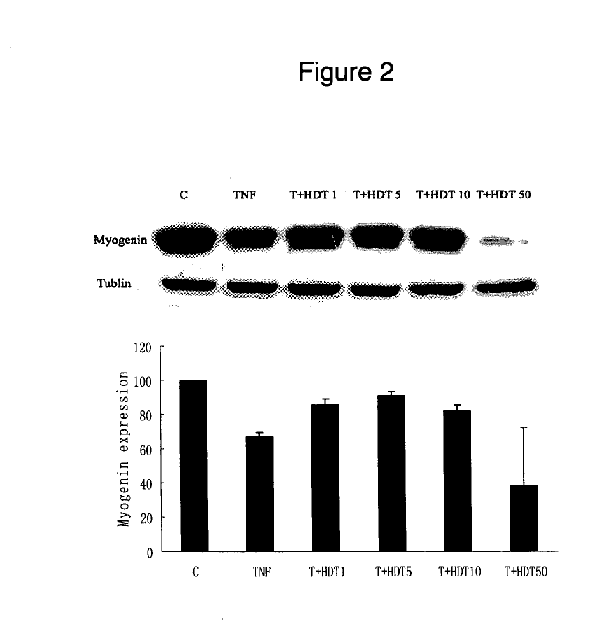

As shown in FIGS. 1 and 2, Western blotting was used to obtain an estimate of the actual increase in muscle specific proteins MHC and myogenin caused by hydroxytyrosol treatment. Hydroxytyrosol showed an increase on MHC protein at 1.0 μM (FIG. 1), and hydroxytyrosol increased myogenin expression at 0.1 μM, and 1.0 μM (FIG. 2).

### Effects of Hydroxytyrosol on CK Activities in C2C12 Cells During the Myogenic Differentiation Induced by TNF-α.

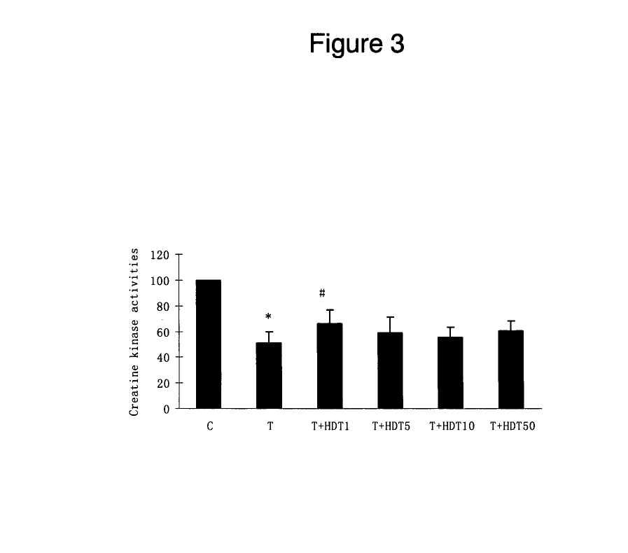

As CK is a muscle cell-specific enzyme, we examined in vitro whether hydroxytyrosol could increase the CK activities in the C2C12 cells during myogenic differentiation. As shown in FIG. 3, the CK activities was significantly increased with hydroxytyrosol at 1.0 μM (p<0.05).

Effects of Hydroxytyrosol on PGC-1α Protein Level in Differentiating C2C12 Cells Treated with TNF-α.

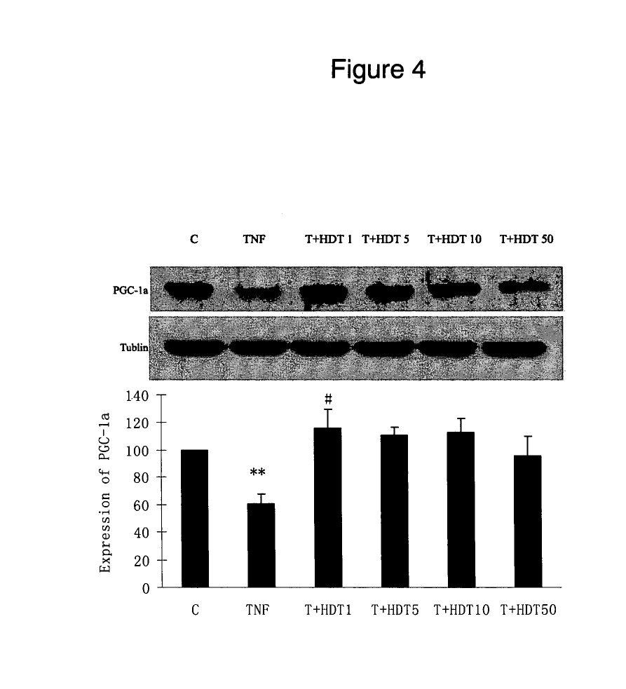

The PGC-1α is a coactivator that promotes mitochondrial biogenesis. As shown in FIG. 4, hydroxytyrosol significantly increased the expression of PGC-1α at 1.0 μM (p<0.05).

Effects of Hydroxytyrosol on Expression and Activities of Mitochondrial Complex I and Complex II in Differentiating C2C12 Cells Treated with TNF-α.

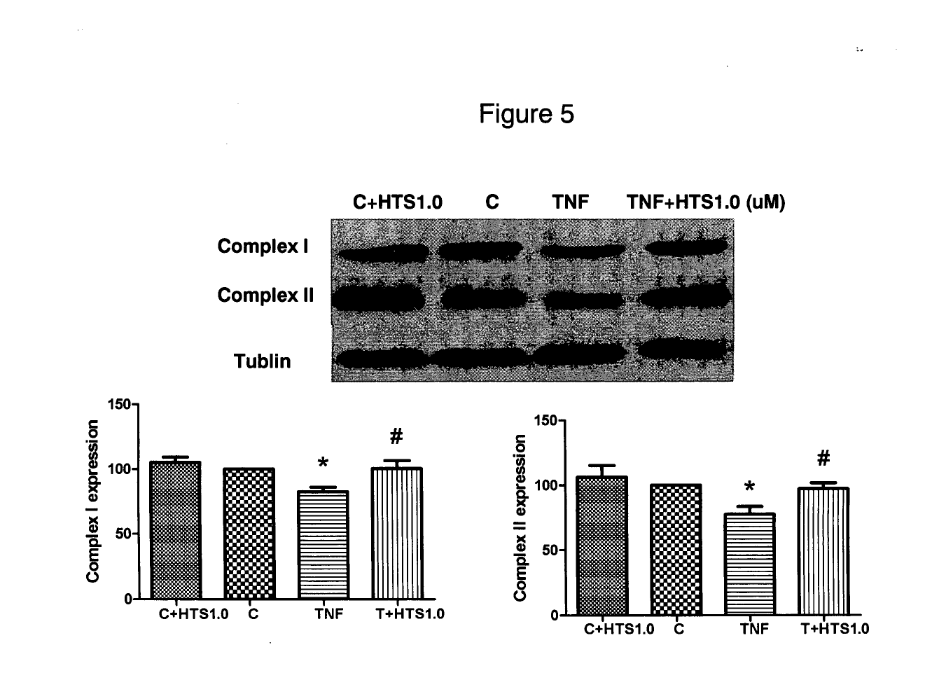

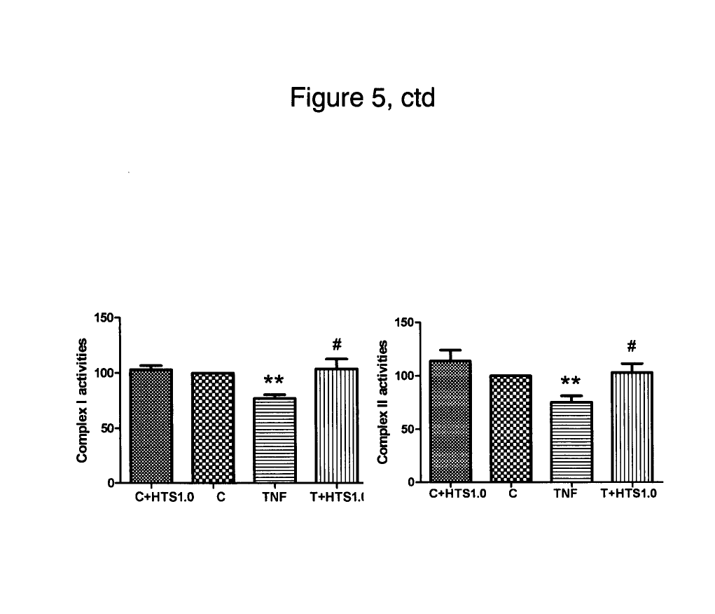

As shown in FIG. 5, hydroxytyrosol increased the expression and activities of mitochondrial complex I and complex II expression at 1.0 μM.

Effects of Hydroxytyrosol on the Differentiation of C2C12 Cells Treated with TNF-α.

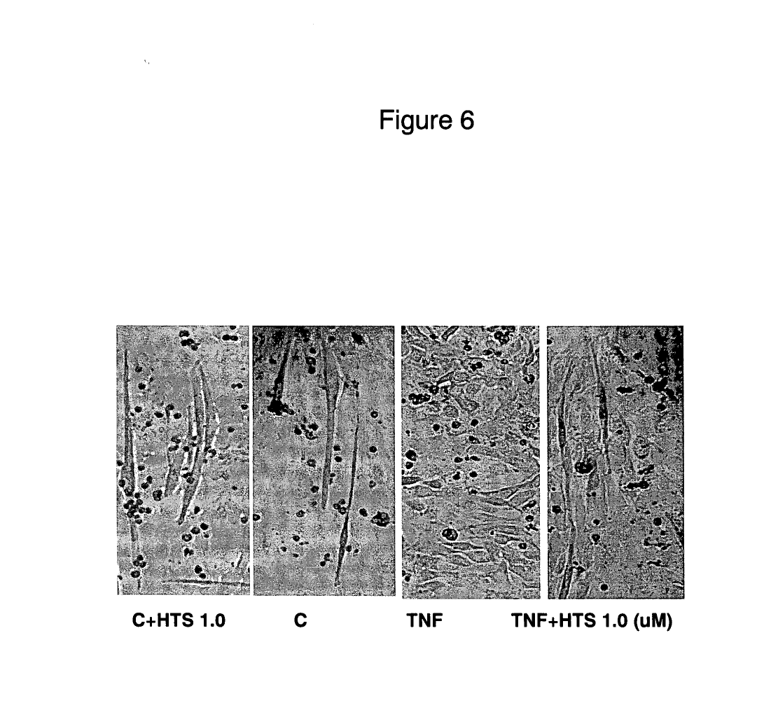

As shown in FIG. 6, hydroxytyrosol increased the expression and activities of mitochondrial complex I and complex II expression at 1.0 μM.

### Effects of Hydroxytyrosol on ROS Level and Activation of NF-kB, JNK in C2C12 Cells During the Myogenic Differentiation Induced by TNF-α.

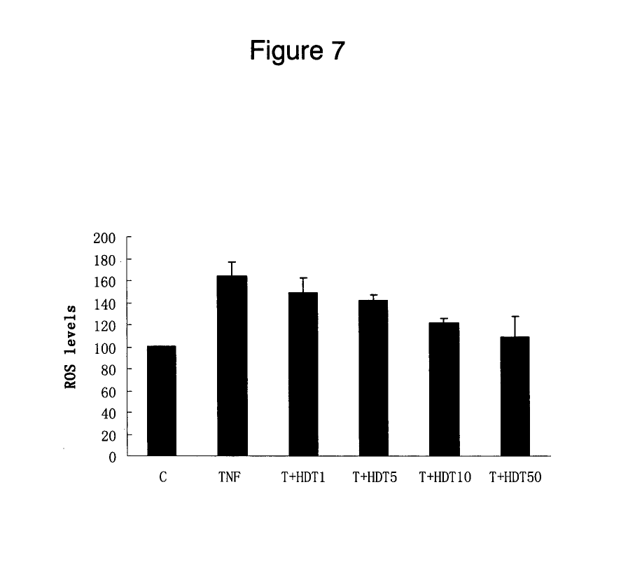

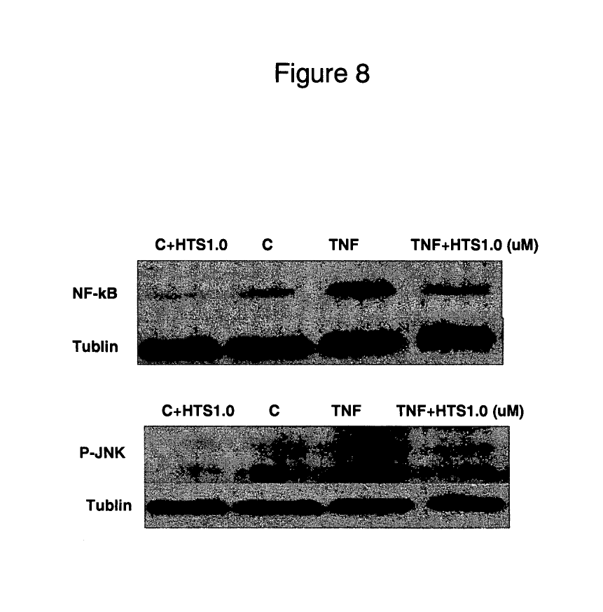

It can been seen from the FIGS. 7 and 8 that TNF-α elevated ROS levels and activated NF-kB, JNK in C2C12 cells. Treatment with hydroxytyrosol inhibited ROS production, and NF-kB as well as JNK activation.

### Example 2

A 29 year old male fitness enthusiast drinks a fitness water (such as Propel, Mizone or similar) comprising 50 mg hydroxytyrosol per 8 fl oz every day for 1 month before and during his regular resistance exercise. The hydroxytyrosol-containing fitness water helps him do 5% more exercise work before developing DOMS.

### Example 3

A sports supplement contains 100 mg hydroxytyrosol per daily dose.

### Example 4

Anti-PPARGC1A and Drp1 antibodies were purchased from Santa Cruz Biotechnology (Santa Cruz, Calif., USA); Anti-GAPDH LC3B, beclin1, p53, p21 were from Cell Signaling Technology (MA, USA); Reverse Transcription System kit was from Promega (Mannheim, Germany); SYBR was from Takara (Otsu, Shiga, Japan); Mn-SOD, Tfam, Atrogin, MuRF1 and 18SrRNA were synthesized by Baiaoke Biotech (Beijing, China); Hydroxytyrosol—pure and as a 15% Hydroxytyrosol powder from of an olive extract—was from DSM Nutritional Products Ltd., Switzerland. TRIzol and other reagents were from Invitrogen (Carlsbad, USA).

### Animals

Sprague-Dawley male rats were purchased from a commercial breeder (SLAC, Shanghai). The rats were housed in a temperature—(22-28° C.) and humidity—(60%) controlled animal room and maintained on a 12-h light/12-h dark cycle (light on from 08:00 a.m. to 08:00 p.m.) with free access to food and water throughout the experiments. Female rats weighing 180-200 g were used. At the beginning of experiments, male rats were selected by one week running exercise at low speed (10 m/min, 20 min/day) and those high exercise activity rats were chosen for the experiments.

### Endurance Exercise Procedure

Rats were randomly divided into four groups: Sedentary, Sedentary with HT supplement (25 mg/kg/day), Endurance exercise and Endurance exercise with HT supplement (25 mg/kg/day). HT was administrated by gavage 45 min before exercise program for each animal. Rats were run on a motorized treadmill at a speed of 20 m/min and a grade of 5° for 1 hour per day and 6 days per week. After 8 weeks exercise, endurance capacity was measured by treadmill running to exhaustion at a speed of 30 m/min and a grade of 5°. Exhaustion was defined as the inability to maintain running and avoid sound and light irritation.

### Isolation of Skeletal Muscle Mitochondria

The soleus muscle was removed from each leg. A first portion was frozen in liquid N₂ and used for total RNA and protein extraction. A second portion was used immediately for mitochondrial isolation. Soleus muscles were trimmed off fat and connective tissue, chopped finely with a pair of scissors, and used for mitochondrial isolation.

### Assay for the Activities of Mitochondrial Complexes

NADH-ubiquinone reductase (complex I), succinate-CoQ oxidoreductase (complex II), ubiquinol cytochrome c reductase (complex III), Mg²⁺-ATPase (complex V) were measured spectrometrically using conventional assays.

### C2C12 Cell Differentiation

Mouse C2C12 myoblasts were purchased from ATCC (Manassas, Va., USA) and maintained in Dulbecco's modified Eagle's medium supplemented with 10% fetal bovine serum (Invitrogen) at a confluence of 60-70%. To initiate differentiation, cells were allowed to reach 100% confluence, and medium was changed to Dulbecco's modified Eagle's medium containing 2% horse serum (Invitrogen) and changed every 2 days. Full differentiation with myotube fusion and spontaneous twitching was observed at 8 days.

### Western Blot Analyses

Samples were lysed with Western and IP lysis buffer (Beyotime, Jiangsu, China). The lysates were homogenized and the homogenates were centrifuged at 13,000 g for 15 min at 4° C. The supernatants were collected and protein concentrations were determined with the BCA Protein Assay kit (Pierce 23225). Equal aliquots (20 μg) of protein samples were applied to 10% SDS-PAGE gels, transferred to pure Nitrocellulose Membranes (PerkinElmer Life Sciences, Boston, Mass., USA), and blocked with 5% non-fat milk. The membranes were incubated with anti-Mfn1, anti-Mfn2, anti-Drp1, anti-PGC-1, anti-MnSOD, anti-pErk1/2, anti-Erk1/2, anti-p-JNK, anti-JNK (1:1000 Santa Cruz), anti-Atg3, anti-Atg7, anti-LC3B, anti-Complex I, II, III, IV, V, anti-β-actin (1:10000 Sigma) at 4° C. overnight. Then the membranes were incubated with anti-rabbit or anti-mouse antibodies at room temperature for 1 hour. Chemiluminescent detection was performed by an ECL Western blotting detection kit (Pierce). Nuclear and cytoplasmic Nrf2 were prepared with Nuclear and Cytoplasmic Protein Extraction Kit (Beyotime Institute of Biotechnology, China) and tested by Western blot.

### Real Time PCR

Total RNA was extracted from 30 mg of tissue using Trizol reagent (Invitrogen) according to the manufacturer's protocol. 2 μg of RNA was reverse transcribed into cDNA. Quantitative PCR was performed using a real-time PCR system (Eppendorf, Germany). Reactions were performed with SYBR-Green Master Mix (TaKaRa, DaLian, China) with specific primers. The primers were as follows:

atrogin-1:
(SEQ. ID NO. 1)
5-CCATCAGGAGAAGTGGATCTATGTT-3 (forward)
and
(SEQ ID NO. 2)
5-GCTTCCCCCAAAGTGCAGTA-3 (reverse);
MuRF1:
(SEQ. ID NO. 3)
5-GTGAAGTTGCCCCCTTACAA-3 (forward)
and
(SEQ. ID NO. 4)
5-TGGAGATGCAATTGCTCAGT-3 (reverse);
FoxO3a:
(SEQ. ID NO. 5)
5-TGCCGATGGGTTGGATTT-3 (forward)
and
(SEQ. ID NO. 6)
5-CCAGTGAAGTTCCCCACGTT-3 (reverse);
18SRNA:
(SEQ. ID NO. 7)
5-CGAACGTCTGCCCTATCAACTT-3 (forward)
and
(SEQ. ID NO. 8)
5-CTTGGATGTGGTAGCCGTTTCT-3 (reverse);
Tfam:
(SEQ. ID NO. 9)
5-AATTGCAGCCATGTGGAGG-3 (forward)
and
(SEQ. ID NO. 10)
5-CCCTGGAAGCTTTCAGATACG-3 (reverse);
Mn-SOD:
(SEQ. ID NO. 11)
5-TGCTCTTCAGCCTGCACTG-3 (forward);
and
(SEQ. ID NO. 12)
5-GGTTCTCCACCACCCTTAG-3 (reverse).

### Statistical Analysis

All data were reported as mean±SEM. Statistical analysis was performed using Graph Prism 4.0.3 software (Graph Pad Software, Inc., San Diego, Calif.). Student's t test was used to compare sedentary and endurance exercise. A one-way ANOVA was employed to detect differences among endurance exercise, sedentary with hydroxytyrosol and endurance exercise with hydroxytyrosol. For all tests the significant level was set at p<0.05.

### Results

### LTE (Long Term Exercise) on Endurance Capacity and Muscle Atrophy and the Effect of HT Supplement

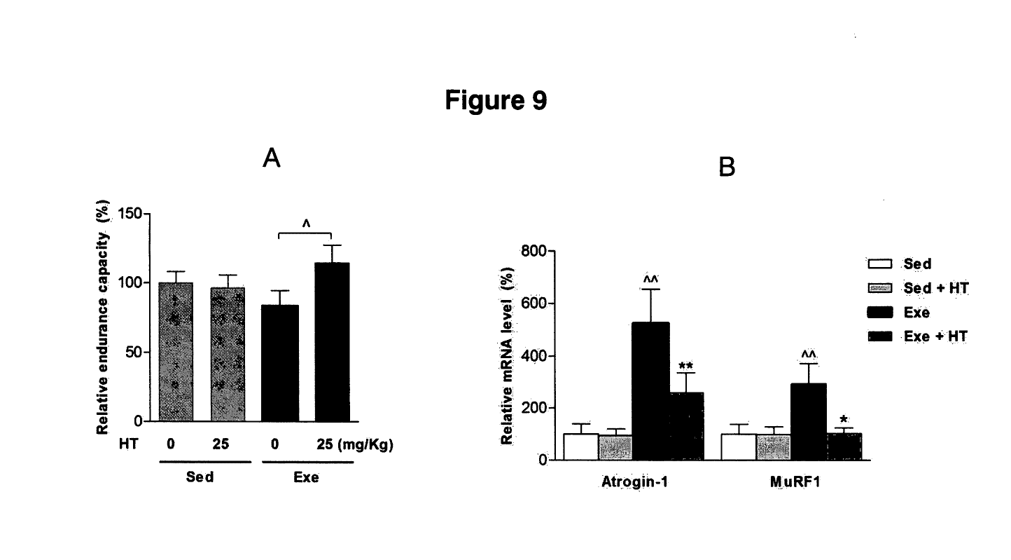

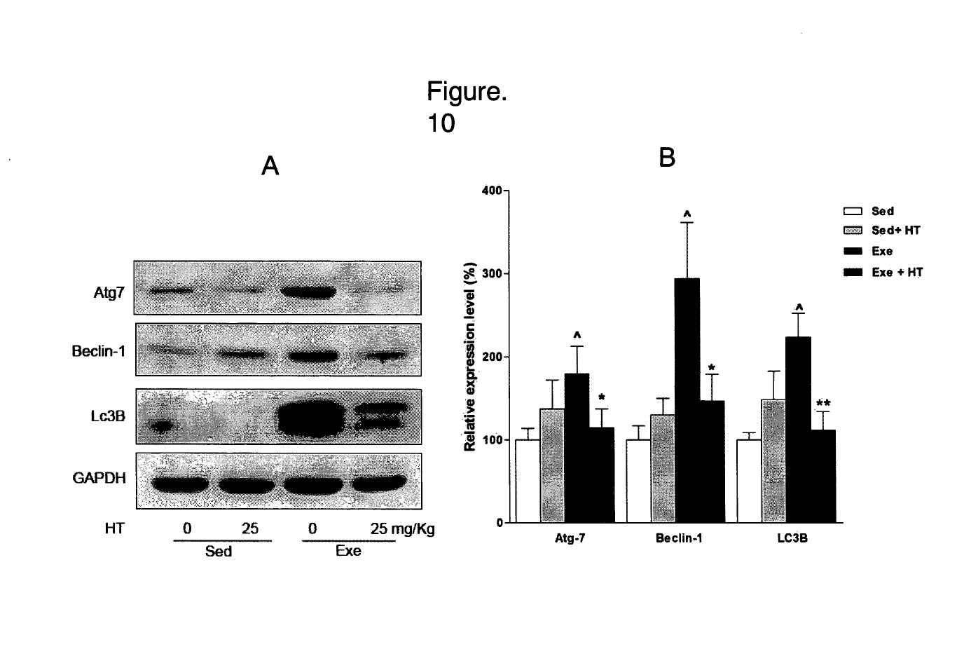

We performed a LTE program with rats and studied the effects of HT supplement on physical performance and the underlying mechanism of mitochondrial dynamics. We showed that LTE was prone to reduce endurance capacity, and HT supplement was sufficient to improve endurance capacity of exercise rats by 35% without any effect on sedentary rats (FIG. 9A). We also found that LTE significantly increased Atrogin-1 and MuRF1 mRNA content which are two well known muscle atrophy markers (FIG. 9B). Further, HT supplement significantly inhibited muscle atrophy progression (FIG. 9B).

### LTE on Activation of Autophagic Pathway and Effect of Ht Supplement

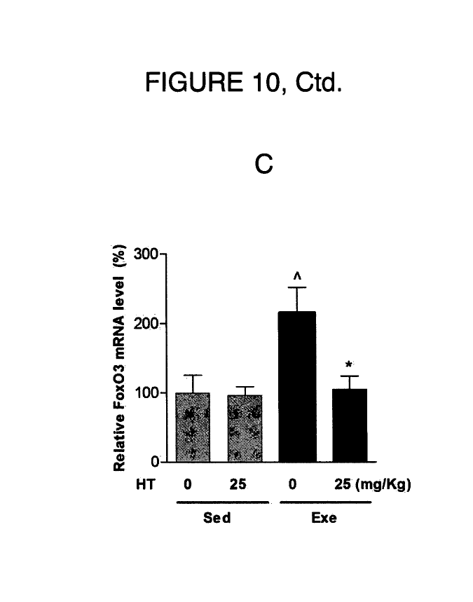

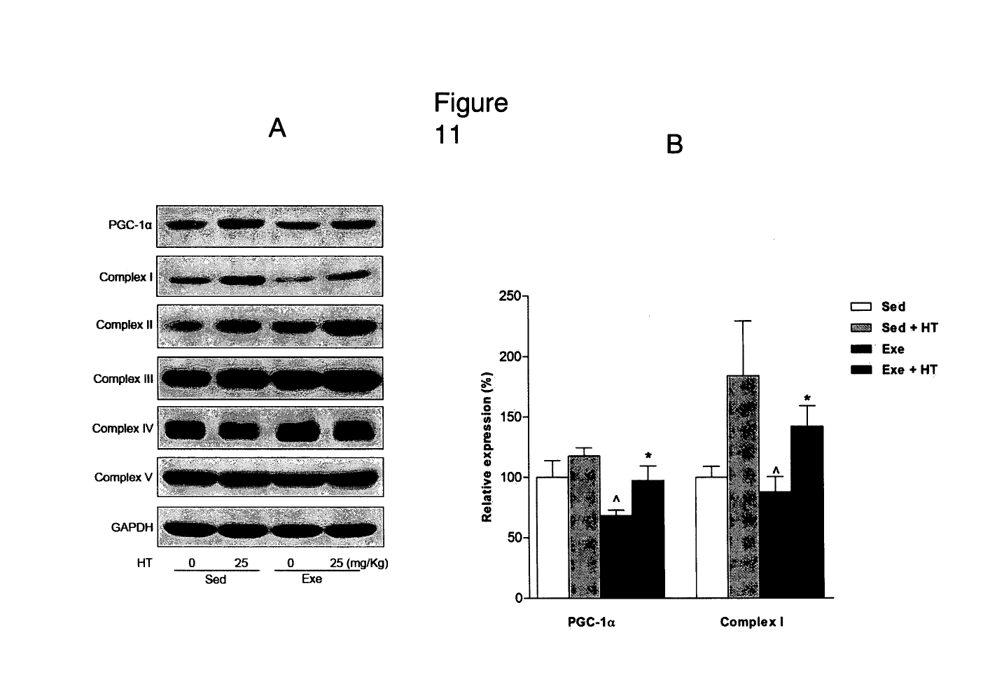

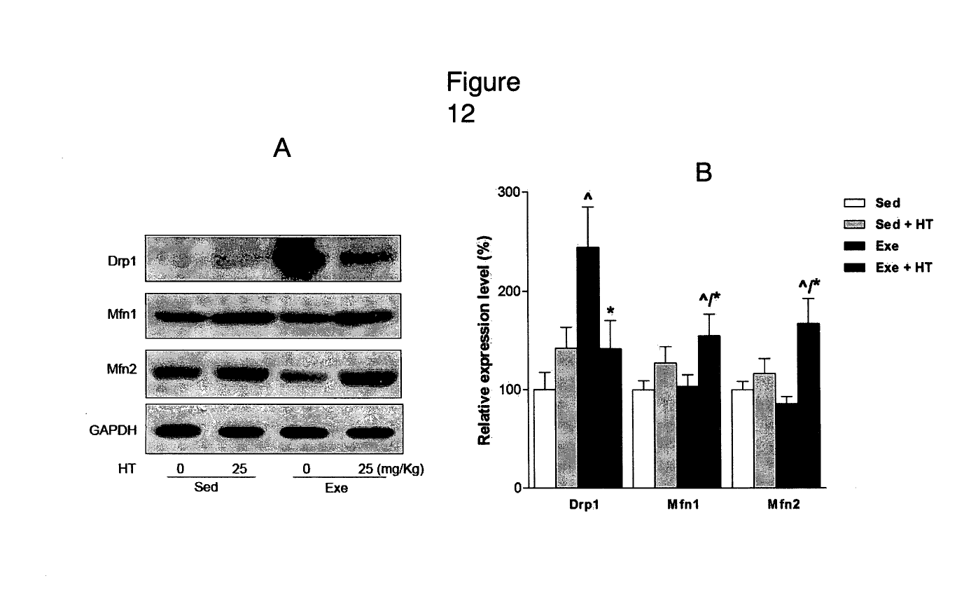

Given the critical role of muscle atrophy regulation, autophagy activation was determined with skeletal muscle protein. Western blot results showed that autophagy related proteins Atg7, Beclin-1 and LC3 were highly induced by LTE (FIG. 10A, B). Furthermore, the mRNA level of a well known autophagy upstream regulator FoxO3 was also increased by LTE (FIG. 10C). All of these changes were efficiently eliminated by HT supplement in LTE rats (FIG. 10 A, B, C). Thus, another aspect of this invention is a method of reducing muscle atrophy by administering HT and observing reduced muscle atrophy. This can be observed by various methods, i.e. noticing that muscle remains intact, and/or by measuring these biochemical markers.

### LTE on Mitochondria Dynamic Remodeling and Effect of HT Supplement

Moderate exercise was known to induce mitochondrial biogenesis through PGC-1α activation. In our LTE study, we found that LTE decreased PGC-1α and complex I subunit expression and HT supplement inhibited the decrease in both PGC-1α and complex I subunit expressions (FIG. 11A, B). Complex II, III, IV, V subunits were not affected by LTE or HT supplement. Mitochondrial DNA copy and NRF1 mRNA level was also not affected by LTE or HT supplement (FIG. 11C). Interestingly, the mRNA level of mitochondrial transcription factor A (Tfam) was found to be increased by LTE and inhibited by HT supplement (FIG. 11C).

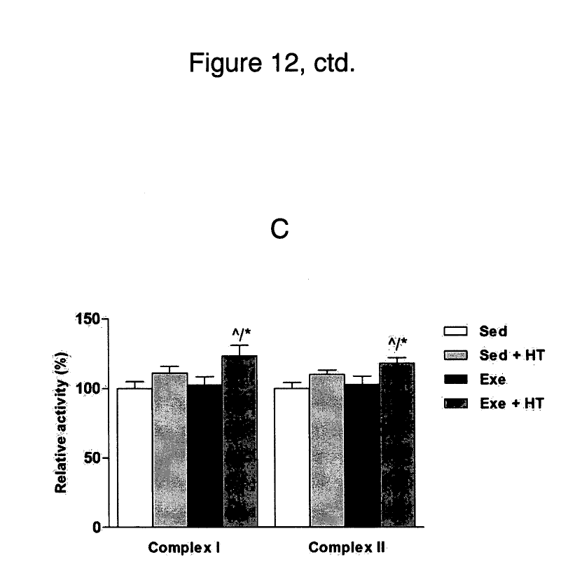

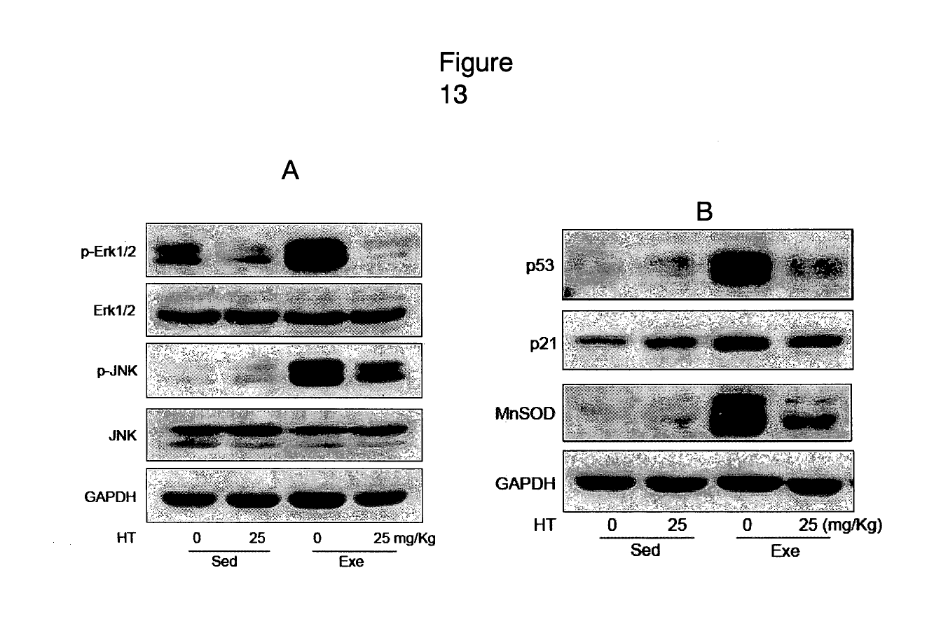

Despite of mitochondrial biogenesis through PGC-1α regulation, mitochondria homeostasis was also regulated by fusion and fission reactions which lead to a continuous remodeling of the mitochondrial network (Bo et al., 2010. Ann N Y Acad Sci 1201, 121-128). In the present study, we found that LTE significantly increased expression of mitochondrial fission related protein Drp1 without affecting mitochondrial fusion related proteins Mfn1, Mfn2 (FIG. 12 A, B). HT supplement inhibited LTE-induced increase in Drp1 expression and also significantly increased Mfn1 and Mfn2 expressions in LTE rats (FIG. 12A, B). Meanwhile, mitochondrial complex I and II activities were found increased by HT supplement in LTE rats (FIG. 12C).

### LTE on Oxidative Pathways and Effect of HT Supplement

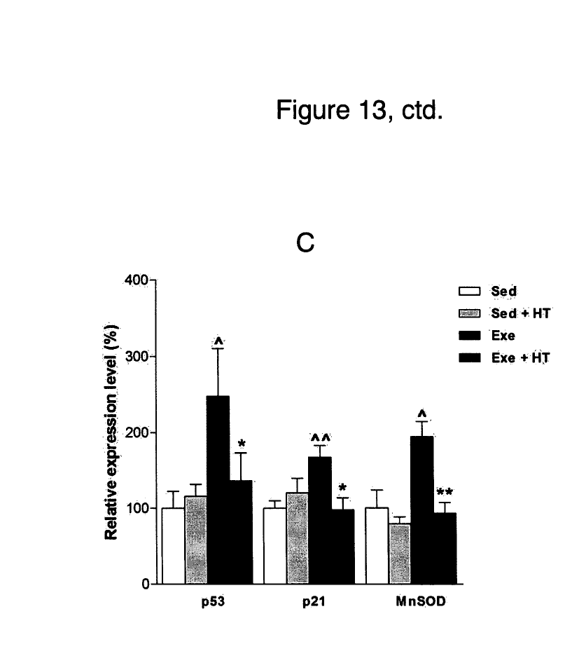

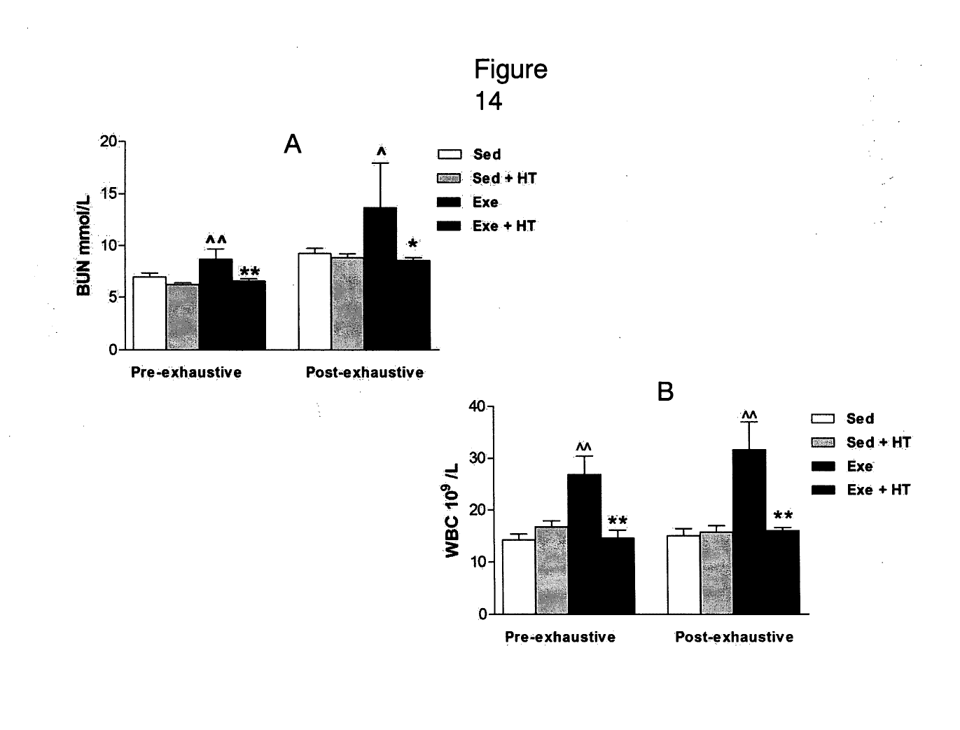

We examined the oxidative status induced by LTE, and found that Erk1/2 and JNK were activated by LTE (FIG. 13A). Meanwhile, oxidative response proteins p53, p21, MnSOD were upregulated by LTE, and also HT supplement, though having no effect on GSH and MDA (not shown), significantly inhibited the LTE-induced increase in Erk1/2, JNK, P53, p21, and MnSOD, respectively (FIGS. 13B and 13C).

### LTE on Renal Function and Immune System and Effect of HT Supplement

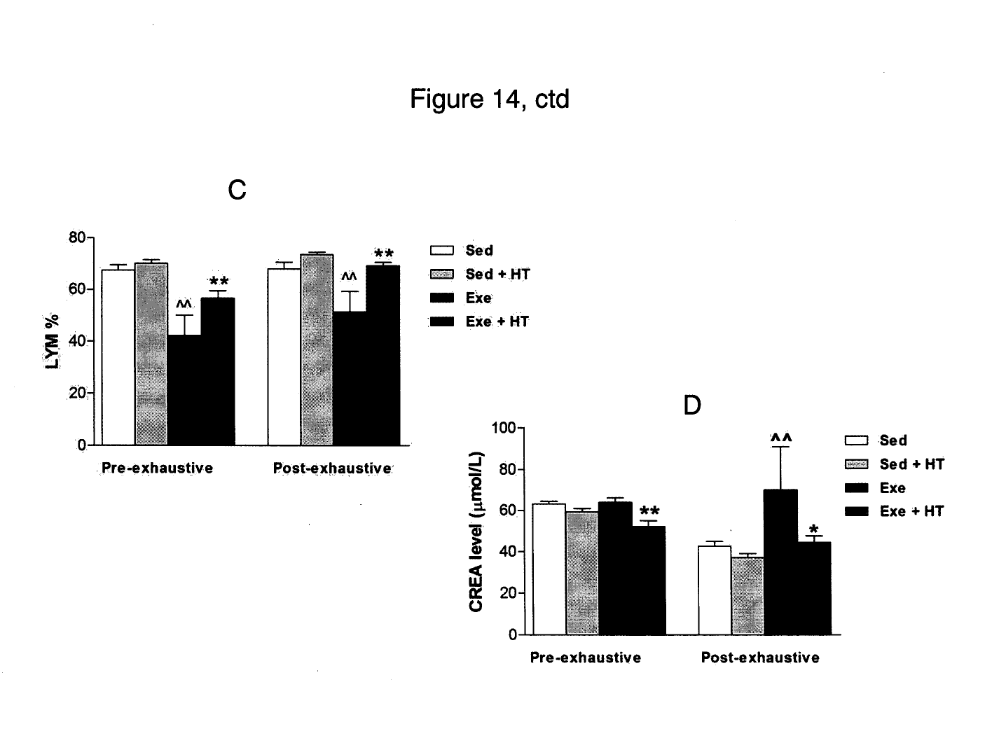

Blood samples were taken before and after endurance capacity test after 8 week LTE. BUN level and WBC number were significantly increased and LYM number was significantly decreased in both pre- and post-exhaustive exercise. All of these changes were restored to normal level by HT supplement (FIG. 14A, B, C). CREA level was not affected in pre-exhaustive animals but significantly increased in the post-exhaustive animals and HT supplement significantly inhibited this increase and also showed reducing effect on CREA level in pre-exhaustive animals (FIG. 14D).

Exercise-induced adaptations in muscle are highly specific and dependent upon the type of exercise, as well as its frequency, intensity, and duration during the exercise. In our study, we performed an LTE program to exhaustion in rats. We showed that LTE was prone to decrease endurance capacity. Since skeletal muscle function is the major component that affects exercise ability, our study was mainly focused on the skeletal muscle adaptation during the LTE and HT supplement.

Autophagy is a catabolic process involving the degradation of a cell's own components through the lysosomal machinery, and helps to maintain a balance between synthesis and degradation of cellular components. However, the role and regulation of the autophagic pathway in skeletal muscle is still not completely understood. Autophagy has been found to be able to clear damaged proteins and organelles to maintain muscle function. Masiero et al. (Masiero et al., 2009 Cell Metab 10, 507-515) reported that Atg7 knock-out—ATG7 being the crucial autophagy gene—results in profound muscle atrophy and age-dependent decrease in muscle force. Very recently, Mammucari et al. (Mammucari et al., 2007 Autophagy 4, 524-526) reported that overexpression of constitutively active FoxO3 could activate autophagy, while knocking down the critical gene LC3 by RNAi partially prevented muscle loss. Consistent with this report, we found that both the muscle atrophy markers Atrogin-1, and MuRF1 as well as the autophagy markers Atg7, Beclin-1, LC3, and FoxO3 were highly induced by LTE. We concluded that LTE to exhaustion could activate autophagy progress to contribute to muscle atrophy and decreased endurance capacity.

Mitochondria are highly dynamic organelles in the production of energy, which are crucial for metabolic activity in skeletal muscle. It is well established that regular exercise activates PGC-1α, thereby inducing nuclear respiratory factors (NRF1 and 2) which in turn promote the expression of numerous nuclear genes encoding mitochondrial proteins as well as mitochondrial transcription factor A (Tfam), leading directly to stimulation of mitochondrial DNA replication and transcription. Furthermore, it is known that that PGC-1α is activation during exercise. Interestingly, in our current studies, LTE decreased PGC-1α and complex I subunit expression instead of enhancing as observed in the prior art. Mitochondrial DNA copy was not affected, except that Tfam mRNA level was increased. While not wishing to be bound by theory, it might be possible that under LTE, the muscle damage is so severe that it suppresses mitochondrial biogenesis. Consistent with a severe muscle damage, we found that LTE activated the stress-activated protein kinases Erk1/2 and JNK, and their molecular targets p53, p21 and MnSOD. Higher levels of p53 and p21 protein are indicative of cell cycle arrest, and are counterproductive for muscle growth and differentiation. In addition, mitochondrial fusion and fission processes were also sensitive to various physiological and pathological stimuli. Acute exercise was reported to decrease mitochondrial fusion and increase mitochondrial fission (Bo et al., 2010, supra). Inhibition of mitochondrial fission prevented muscle loss during fasting, and induction of mitochondrial fission and dysfunction activated an atrophy program (Romanello et al., 2010 EMBO J 29, 1774-1785). Consistent with these studies, we found that under LTE, mitochondrial fission was activated and the activation might accelerate mitochondrial dysfunction. Increased CREA levels after LTE are also indicative of severe muscle damage.

To further study the effect of LTE and how it is unfluenced by HT, we tested BUN, LYM, and WBC numbers, which represent immune system function.

The results implicated that both musculature and the immune system were stressed during the LTE program.

1. A method of maintaining or increasing muscle differentiation or regeneration after strenuous physical exercise or under conditions where muscle is chronically inflammed, comprising administering an effective amount of hydroxytyrosol (HT) to a mammal, and observing a muscle differentiation effect.

2. A method according to claim 1 wherein the muscle differentiation effect is a lessening of Delayed Onset of Muscle Soreness (DMOS).

3. A method according to claim 1 wherein the physical exercise is sufficient to cause inflammation in the absence of HT.

4. A method according to claim 1 wherein the mammal is a human athlete.

5. A method according to claim 1 wherein the hydroxytyrosol is present in an olive juice extract.

6. A method according to claim 1 wherein the muscle which is chronically inflammed is due to sarcopenia.

7. Use of hydroxytyrosol in the production of a nutraceutical or food product for the use of maintaining or increasing muscle differentiation or regeneration after strenuous physical exercise or when the muscle is chronically inflammed.

8. Use according to claim 7 wherein the muscle differentiation results in a lessening of Delayed Onset of Muscle Soreness (DMOS).

9. Use according to claim 7 wherein the physical exercise is sufficient to cause inflammation in the absence of HT.

10. Use according to claim 7 wherein the nutraceutical or food product is an olive juice extract.

11. Use according to claim 7 wherein the nutraceutical or food product is for consumption by an elite human athlete.
